(19) United States
(12) Patent Application Publication
(10) Pub. No.: US 2005/0277817 A1
(43) Pub. Date: Dec. 15, 2005

(54) Noninvasive measurement system for monitoring activity condition of living body

(75) Inventor: Hideo Eda, Tokyo (JP)

Correspondence Address: BIRCH STEWART KOLASCH & BIRCH, PO BOX 747, FALLS CHURCH, VA, 22040-0747, US

(73) Assignee: National Institute of Information & Communications Technology, Incorporated Administrative Agency, Tokyo (JP)

(21) Appl. No.: 11/184,788

(22) Filed: Jul. 20, 2005

(30) Foreign Application Priority Data
Dec. 3, 2003 (JP) 2003-404678

(51) Int. Cl. A61B 5/00

(52) U.S. Cl. 600/315

(57) ABSTRACT

A noninvasive measurement system for monitoring activity condition of a living body is provided, which can accurately monitor activity condition of a desired region of a living body. The means for measuring activity information is equipped with the light emitting source unit 3 to emit a light beam toward a living body, the light detecting unit 4 to receive the light beam scattered by blood present in the head 10, the temporary operation unit 1A to compute temporary activity condition of blood in the head 10 by performing arithmetical operation of signals of the light beam to be received in the light detecting unit 4, the superficial activity operation unit to compute activity condition of blood in the scalp 14, and the correction operation unit 1B to compute corrected activity condition of the brain 11 by correcting temporary activity condition of the head 10 that is computed in the temporary operation unit 1A based on computed activity condition of blood in the scalp 14 in the superficial activity operation unit. Further, the means for measuring positional information is equipped with the positional information collecting unit to monitor positional information of the brain 11 in the head 10, and the memory unit 8 to memorize positional information of the brain 11 obtained in the positional information collecting unit.

### BACKGROUND OF THE INVENTION

1. Field of the Invention

The present invention relates to a noninvasive measurement system for monitoring activity condition of a living body, equipped with means for measuring activity information for monitoring activity condition of a desired region of a living body and means for measuring positional information for monitoring positional information of a desired region of a living body.

2. Description of the Related Art

As shown in FIG. 2, a conventional noninvasive measurement system for monitoring brain activity condition (a noninvasive measurement system for monitoring activity condition of a living body) C is equipped with a light emitting source unit 23 for emitting a light beam R1 toward a human head (a living body) 30; a light detecting unit 24 receiving a light beam R2, which is scattered by blood present in the head 30; and an operation unit 21, which computes activity condition of the brain (a desired region) 31 in the head 30 by performing arithmetical operation of signals of the light beam R2 to be received in the light detecting unit 24. In FIG. 2, code 32 indicates cerebrospinal fluid, and code 33 indicates cranium.

In addition, code 22 indicates an I/O unit, which is an input-output device for performing input and output of data set by the operation unit 21, and code 25 indicates an A/D converter unit, which is a converter for converting analog signals to digital signals.

To monitor activity condition of the brain 31 using a noninvasive measurement system C, constructed as such, for monitoring brain activity condition, the light emitting source unit 23 and the light detecting unit 24 are set close to a scalp. Near infrared radiation R1 having three different wave lengths (780, 805 and 830 nm) is emitted from the light emitting source unit 23 to irradiate the head 30. Then reflected light R2 is received. The reflected light R2 is a part of the near infrared radiation R1, which is scattered by hemoglobin of blood present in the head 30 and arrives at the light detecting unit 24.

Light intensity of the reflected light R2 varies depending on activity condition of hemoglobin in the brain 31. Consequently, activity condition of the brain 31 could be measured by performing the prescribed computation in the operation unit 21 by measuring the reflected light R2.

In such a conventional noninvasive measurement system C for monitoring brain activity condition, there were such problems as the near infrared radiation R1 picked up not only information of hemoglobin present in the brain 31 but also that of hemoglobin present in the scalp 34. Consequently, the reflected light R2, which is received in the light detecting unit 24, shows mixed information from hemoglobin present in the brain 31 and hemoglobin present in the scalp 34, and has often created problems of inaccurate monitoring of activity of the brain 31.

In accordance with teaching herein, an aspect of the present invention is to provide a noninvasive measurement system for monitoring activity condition of a living body, which can accurately monitor activity condition of a desired region of a living body.

### SUMMARY OF THE INVENTION

The invention according to claim 1 is a noninvasive measurement system for monitoring activity condition of a living body, equipped with means for measuring activity information to monitor activity condition of a desired region of a living body, and means for measuring positional information to monitor positional information of a desired region of a living body, characterized that the means for measuring activity information is equipped with a light emitting source unit to emit a light beam toward a living body; a light detecting unit to receive a light beam scattered by blood present in a living body; a temporary operation unit to compute temporary activity condition of a desired region by performing arithmetical operation of signals of the light beam to be received in the light detecting unit; a superficial activity operation unit to compute activity condition of the surface layer region of a living body; and a correction operation unit to compute corrected activity condition of a desired region by correcting temporary activity condition of a desired region of a living body that is computed in the temporary operation unit based on computed activity condition of the surface layer of a living body in the superficial activity operation unit; and the means for measuring positional information is equipped with a positional information collecting unit to monitor positional information of a desired region in a living body, and a memory unit to memorize positional information of a desired region obtained in the positional information collecting unit.

The invention according to claim 2 is characterized that the superficial activity operation unit is a laser Doppler blood flowmeter in a noninvasive measurement system for monitoring activity condition of a living body according to claim 1.

The invention according to claim 3 is characterized that the positional information collecting unit is magnetic resonance imaging (MRI) equipment in a noninvasive measurement system for monitoring activity condition of a living body according to claim 1.

By the invention according to claim 1, since it is a noninvasive measurement system for monitoring activity condition of a living body, equipped with means for measuring activity information to monitor activity condition of a desired region of a living body, and means for measuring positional information to monitor positional information of a desired region of a living body, and the means for measuring activity information is equipped with a light emitting source unit to emit a light beam toward a living body, a light detecting unit to receive the light beam scattered by blood present in a living body, a temporary operation unit to compute temporary activity condition of a desired region in a living body by performing arithmetical operation of signals of the light beam to be received in the light detecting unit, a superficial activity operation unit to compute activity condition of the surface layer region of a living body, and a correction operation unit to compute corrected activity condition of a desired region by correcting temporary activity condition of a desired region of a living body that is computed in the temporary operation unit based on computed activity condition of the surface layer of a living body in the superficial activity operation unit, reflected light received in the light detecting unit can be separated into reflected light received from blood present in a desired region of a living body and reflected light received from blood present in the surface layer region of a living body, and a noninvasive measurement system for monitoring activity condition of a living body, enabling accurate monitoring of activity condition of a desired region of a living body, can be provided.

In addition, since the means for measuring positional information is equipped with the positional information collecting unit to monitor positional information of a desired region in a living body, and the memory unit to memorize positional information of a desired region obtained in the positional information collecting unit, positional information of a desired region in a living body can be monitored, and at the same time positional information can be memorized and compared with data measured before.

Consequently, since activity condition of a living body can be monitored by combination of activity information and positional information and highly accurate diagnosis can be performed, a noninvasive measurement system for monitoring activity condition of a living body, enabling accurate monitoring of activity condition of a desired region of a living body, can be provided.

By the invention according to claim 2, since the superficial activity operation unit is a laser Doppler blood flowmeter, reflected light received in the light detecting unit can be separated into reflected light received from blood present in the brain and reflected light received from blood present in a scalp and thus, a noninvasive measurement system for monitoring activity condition of a brain, enabling accurate monitoring of activity condition in a brain, can be provided.

By the invention according to claim 3, since the positional information collecting unit is magnetic resonance imaging (MRI) equipment, a noninvasive measurement system for monitoring activity condition of a brain, in which positional information in a brain can be monitored and at the same time positional information of a brain can be memorized and compared with data measured before, can be provided.

### BRIEF DESCRIPTION OF THE DRAWINGS

FIG. 1 is a schematic block diagram of a noninvasive measurement system for monitoring activity condition of a brain as an example of a noninvasive measurement system for monitoring activity condition of a living body of the present invention.

FIG. 2 is a schematic block diagram of a noninvasive measurement system for monitoring activity condition of a brain as an example of a conventional noninvasive measurement system for monitoring activity condition of a living body.

### DESCRIPTION OF THE PREFERRED EMBODIMENT(S)

Referring to drawings hereinbelow, the best mode for carrying out the present invention will be described in detail.

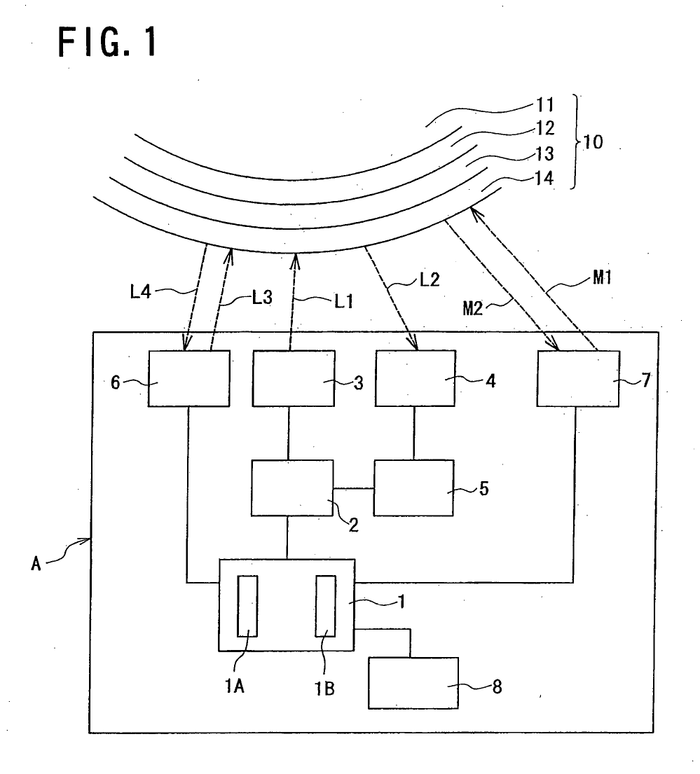

FIG. 1 shows a noninvasive measurement system A for monitoring activity condition of a brain as an example of a noninvasive measurement system of the present invention for monitoring activity condition of a living body.

A noninvasive measurement system A for monitoring activity condition of a brain is composed by an operation unit 1, an I/O unit 2, a light emitting source unit 3, a light detecting unit 4, an A/D converter unit 5, a laser Doppler blood flowmeter 6, magnetic resonance imaging (MRI) equipment 7, a memory unit 8, a monitor and a keyboard (input unit) (not shown).

The operating unit 1 includes a temporary operating unit 1A and a correction operation unit 1B in the inside, and the laser Doppler blood flowmeter 6 has a built-in superficial activity operation unit, a light emitting source unit and a light detecting unit.

The operation unit 1 is one for arithmetic processing of the measured data by the predetermined computing equation and inducing emission of light to the light emitting source unit 3.

The I/O unit 2 is an input-output device for transmitting command to the light emitting source unit 3 from an input device (not shown) or for incorporating measured date from the A/D converter 5 explained hereinbelow. The light emitting source unit 3 is a light emitting device for generating near-infrared ray. The light emitting source unit 3 includes three sets of light emitting devices, each for generating light having three different wavelengths (near-infrared ray at 780, 805 and 830 nm). An optical fiber cable is equipped in the tip of the light emitting source unit 3 not to attenuate optical signals. As for a light source of the light emitting source unit 3, a semiconductor laser, a halogen lump and an optical filter, and LED can be used. Since the light emitting source unit is used for computing two components of oxygenated hemoglobin and deoxygenated hemoglobin, at least two or more wavelengths may preferably be used.

The light detecting unit 4 is a device equipped with a light receiving element for converting reflected light from the head 10 to an electric signal. The light detecting unit 4 has a photomultiplier with function for amplifying optical signals or built-in avalanche photodiode, and is equipped with the optical fiber cable on the tip.

The A/D converter 5 is a converter for converting analog signals to digital signals. The memory unit 8 is a device to memorize past positional information of a brain of the same subject to be tested, measured by MRI equipment 7 to be explained hereinbelow.

The temporary operation unit 1A is an operation unit to compute activity condition of blood present in the brain 11 by comparing wavelength of the near-infrared ray emitted from the light emitting source unit 3 and wavelength of the near-infrared ray received in the light detecting unit. The correction operation unit 1B is an operation unit wherein temporary activity condition of blood present in the brain 11, which is computed in the temporary operation unit 1A, is corrected by comparing with activity condition of blood present in the scalp 14, which is computed in the superficial activity operation unit located in the laser Doppler blood flowmeter 6 to be explained hereinbelow, and corrected activity condition of blood present in the brain 11 is computed.

The laser Doppler blood flowmeter 6 (means for measuring superficial activity) is the superficial activity operation unit to continuously measure blood flow of capillary level tissues of the scalp components, to which laser light is irradiated. The laser Doppler blood flowmeter 6 is equipped with a light emitting source unit, a light detecting unit and an A/D converter, in addition to the superficial activity operation unit, and measures change in frequency based on phenomenon that, on irradiation of light, with a lighting pattern generated by constant repetition frequency, to moving objects such as erythrocytes in the components of the scalp 14, wavelength width broader than irradiated repetition frequency can be detected depending on speed of erythrocytes by irradiation on to erythrocytes. Although a laser beam emitted from the tip of the probe penetrates into tissues and is absorbed by repeated scattering and refraction, light collided with erythrocytes which contain hemoglobin is Doppler shifted. Consequently, light scattered by somatic cells is a mixture of light with Doppler shift, generated by hemoglobin in erythrocytes and light without Doppler shift, scattered by static tissues. The mixed light is converted into an electric signal, and is output and displayed as blood flow volume, which is a product of concentration of moving hematocytes and blood flow rate.

Magnetic resonance imaging (MRI) equipment 7 (means for measuring positional information) is equipment, wherein magnetic resonance signals of hydrogen atoms contained in the cranium 13, water and the brain 11 are measured and converted into image, and has a built-in positional information collecting unit to find out the position of the brain 11.

The head 10 composed of the brain 11, cerebrospinal fluid 12, cranium 13 and scalp 14, from the interior portion to the exterior portion, in this order. Light emitted from the light source of the light emitting source unit 3 is scattered mainly by erythrocytes of the brain 11 and the scalp 14 in the head 10 to be received with the light receiving element in the light detecting unit 4.

Consequently, there are two pathways for receiving light from the light emitting source unit 3 to the light detecting unit 4, i.e., one pathway, wherein light arrives at the brain 11 after passing through the scalp 14, cranium 13, cerebrospinal fluid 12, etc., and is subsequently scattered by blood in the brain 11, and is received after passing through the cerebrospinal fluid 12, cranium 13, scalp 14, etc., and the other pathway, wherein light is scattered by blood in the scalp 14 and is received.

For monitoring activity condition of the brain 11 of a subject to be tested using thus constructed noninvasive measurement system for monitoring brain activity condition, the light emitting source unit 3 and the light detecting unit 4 are set by attaching close to the scalp 14. Then an observer inputs measurement conditions into the input unit. After the input operation, measurement conditions are converted from digital signals to analog signals for transmission, and are transmitted to the light emitting source unit 3 via the I/O unit 2. The light emitting source unit 3 recognizes the analog signals, and sequentially emits the analog signals in a form of pulsed three near-infrared rays L1 (e.g. wavelengths of 780, 805 and 830 nm, specified wavelength ±10 nm, and half-width of 5 nm) from the light emitting source unit 3 through the optical fiber cable from the semiconductor laser of the light source in the light emitting source unit 3. The near-infrared rays L1 are reflected in the brain 11 and the scalp 14 in the head 10, and is received in the light detecting unit 4 located at a position different from the light emitting source unit 3, as the reflected light L2. Subsequently, the reflected light is converted from optical signals to electrical signals, that is, analog signals, by a photomultiplier in the light detecting unit 4 and is amplified. Thus amplified optical signals are converted to digital signals by the A/D converter unit 5. The digital signals are transmitted to the temporary operation unit 1A located in the operation unit 1 via the I/O unit 2 to compute temporary activity condition of the brain 11.

On the other hand, to monitor activity condition of the scalp 14 using the laser Doppler blood flowmeter, the near-infrared ray L3 with wavelength of 670 nm is emitted from the light emitting source unit as a light source, and the reflected light L4 is received in the light detecting unit located at a position close to the light emitting source unit. Then, the reflected light is treated through the amplifier and the built-in A/D converter unit in the laser Doppler blood flowmeter 6 and activity condition of the scalp 14 is computed in the superficial activity operation unit, and then transmitted to the operation unit 1.

To monitor positional information of the brain 11 in the head 10 using MRI 7, strong and uniform static magnetic field and variational magnetic field M1 are supplied to the head 10, and the weak electric wave M2, which is generated based on the magnetic resonance phenomenon originated from hydrogen nucleus present in the head 10, is received in positional information collecting unit, then the electric wave signal is digitized, converted to imaging, and stored in the memory unit 8 via the operation unit 1. The stored tomograms of the brain 11 are displayed in a monitor.

Since the position in the brain 11 of the head 10 is changed depending on the posture of the subject to be tested at the measurement, it is necessary to measure the position in the brain with the same posture in advance. For that purpose, previously measured tomograms are stored in the memory unit 8. Since positional information of the brain 11 together with activity condition of blood in the brain 11 can be measured by confirming difference in the position of the brain 11 in comparison with the previously measured tomogram and the tomogram measured in this time, activity condition of the brain 11 can be diagnosed more accurately.

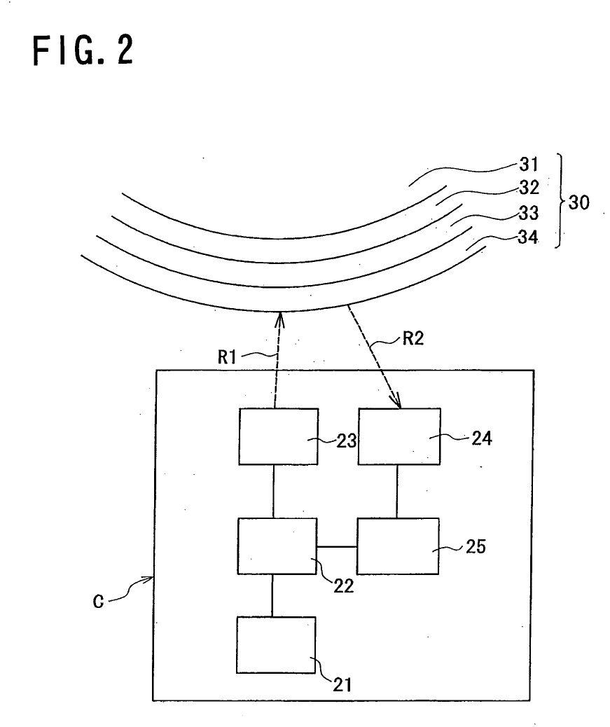

When a conventional noninvasive measurement system for monitoring brain activity condition C is applied to the brain 10 shown in FIG. 2, light emitted from the light emitting source unit 23 contains two measured data including information derived from hemoglobin in the brain 31 and information derived from hemoglobin in the scalp 34. Further, since the effect of the skin blood flow in the scalp 34 is larger than the effect of blood flow in the brain in measurement results, hemoglobin in the scalp causes large measurement error and accurate measurement of activity condition of blood in the brain 31 has been difficult.

In using the laser Doppler blood flowmeter 6 as shown in FIG. 1, only skin blood flow in the scalp 14 can be measured.

Namely, in the operation unit 1, data derived from the temporary operation unit 1A computed in a process through the light emitting source unit 3, and data derived from the superficial activity operation unit computed in a process through the laser Doppler blood flowmeter 6 are computed by the correction operation unit 1B, and as a result, true activity condition of blood in the brain 11 excluding possible effects of the skin blood flow can be observed.

Since an error factor caused by skin blood flow is eliminated and highly precise activity condition in a living body can be measured, the system can be applied for fundamental research on brain functions and examination on brain diseases.

As explained in detail, a noninvasive measurement system for monitoring brain activity condition A (a noninvasive measurement system for monitoring activity condition of a living body) of the present invention is equipped with means for measuring activity information for monitoring activity condition of blood in the head 10 and means for measuring positional information for monitoring positional information of the brain 11 in the head 10.

The means for measuring activity information is equipped with the light emitting source unit 3 to emit a light beam toward a living body, the light detecting unit 4 to receive the light beam scattered by blood present in the head 10, the temporary operation unit 1A to compute temporary activity condition of blood in the head 10 by performing arithmetical operation of signals of the light beam to be received in the light detecting unit 4, the superficial activity operation unit to compute activity condition of blood in the scalp 14, and the correction operation unit 1B to compute corrected activity condition of the brain 11 by correcting temporary activity condition of the head 10 that is computed in the temporary operation unit 1A based on computed activity condition of blood in the scalp 14 in the superficial activity operation unit.

The means for measuring positional information is equipped with the positional information collecting unit to monitor positional information of the brain 11 in the head 10, and the memory unit 8 to memorize positional information of the brain 11 obtained in the positional information collecting unit.

1. A noninvasive measurement system for monitoring activity condition of a living body, equipped with means for measuring activity information to monitor activity condition of a desired region of a living body, and means for measuring positional information to monitor positional information of a desired region of a living body characterized that
the means for measuring activity information is equipped with
a light emitting source unit to emit a light beam toward a living body;
a light detecting unit to receive the light beam scattered by blood present in a living body;
a temporary operation unit to compute temporary activity condition of a desired region by performing arithmetical operation of signals of the light beam to be received in the light detecting unit;
a superficial activity operation unit to compute activity condition of the surface layer region of a living body; and
a correction operation unit to compute corrected activity condition of a desired region by correcting temporary activity condition of a desired region of a living body that is computed in the temporary operation unit based on computed activity condition of the surface layer of a living body in the superficial activity operation unit; and
the means for measuring positional information is equipped with
a positional information collecting unit to monitor positional information of a desired region in a living body, and
a memory unit to memorize positional information of a desired region obtained in the positional information collecting unit.

2. A noninvasive measurement system for monitoring activity condition of a living body according to claim 1, wherein the superficial activity operation unit is a laser Doppler blood flowmeter.

3. A noninvasive measurement system for monitoring activity condition of a living body according to claim 1, wherein the positional information collecting unit is magnetic resonance imaging (MRI) equipment.
